(19) United States
(12) Patent Application Publication
(10) Pub. No.: US 2023/0114715 A1
(43) Pub. Date: Apr. 13, 2023

(54) SEPARATION MEMBRANE COMPLEX AND SEPARATION METHOD

(71) Applicant: NGK Insulators, Ltd., Nagoya-City (JP)

(72) Inventors: Noriyuki OGASAWARA, Nagoya-City (JP); Kenji YAJIMA, Nagoya-City (JP); Makiko ICHIKAWA, Nagoya-City (JP)

(73) Assignee: NGK Insulators, Ltd., Nagoya-City (JP)

(21) Appl. No.: 18/064,364

(22) Filed: Dec. 12, 2022

Related U.S. Application Data
(63) Continuation of application No. PCT/JP2021/014908, filed on Apr. 8, 2021.

(30) Foreign Application Priority Data
Jul. 21, 2020 (JP) 2020-124368

(51) Int. Cl. B01D 63/06; B01D 71/02; B01D 69/02; B01D 69/10; B01D 53/22; B01J 20/18; B01J 20/08; B01J 20/28; C07C 7/144; C07C 7/12

(52) U.S. Cl. CPC B01D 63/066; B01D 71/0281; B01D 69/02; B01D 69/108; B01D 53/228; B01J 20/18; B01J 20/08; B01J 20/28035; B01J 20/28004; B01J 20/28085; B01J 20/2803; B01J 20/28045; C07C 7/144; C07C 7/12; B01D 2325/04; B01D 2325/0283; B01D 2325/02833

(57) ABSTRACT

A separation membrane complex includes a porous support and a separation membrane formed on the support and used to separate fluid. A supply/permeation area ratio obtained by dividing a supply-side surface area by a permeation-side surface area is higher than or equal to 1.1 and lower than or equal to 5.0, the supply-side surface area being the area of a region of the surface of the separation membrane to which fluid is supplied, the permeation-side surface area being the area of a region of the surface of the support from which fluid that has permeated through the separation membrane and the support flows off.

### CROSS-REFERENCE TO RELATED APPLICATION

The present application is a continuation application of International Application No. PCT/JP2021/014908 filed on Apr. 8, 2021, which claims the benefit of priority to Japanese Patent Application No. JP2020-124368 filed on Jul. 21, 2020. The entire contents of these applications are incorporated herein by reference.

### TECHNICAL FIELD

The present invention relates to a separation membrane complex and a separation method using the separation membrane complex.

### BACKGROUND ART

Various studies and developments are currently underway to obtain a zeolite membrane complex by forming a zeolite membrane on a porous support and to use the molecular-sieving function of the zeolite in applications such as separation of specific molecules or adsorption of molecules.

For example, Japanese Patent No. 5937569 (Document 1) discloses a separation membrane structure that includes a porous base material of a honeycomb shape including a plurality of cells, a porous intermediate layer arranged on the surface of the base material in the cells, and a zeolite membrane arranged on the surface of the intermediate layer. Japanese Patent Application Laid-Open No. 2017-80744 (Document 2) discloses a separation membrane structure in which a zeolite membrane is formed by hydrothermal synthesis on the outer peripheral surface of a cylindrical porous support.

In the case of a separation membrane structure as disclosed in Document 1, if the supply-side surface area to which a fluid mixture (e.g., a mixed gas) that is subjected to separation is supplied is excessively smaller than the permeation-side surface area from which fluid that has permeated through the separation membrane structure flows off, the amount of fluid (i.e., flux) that permeates through the separation membrane per unit time may decrease and this may result in decreased efficiency of separation processing and increased processing cost. In the case of a cylindrical separation membrane structure including a zeolite membrane on the outer peripheral surface as disclosed in Document 2, the thickness of a cylindrical base material may decrease and this may result in lower strength of the separation membrane structure.

Moreover, if the aforementioned supply-side surface area is excessively larger or excessively smaller than the permeation-side surface area, the process of burning and removing a structure-directing agent from the zeolite membrane in the production of the separation membrane structure may require an increase in heating temperature in order to burn and remove the structure-directing agent, or may end with insufficient or non-uniform removal of the structure-directing agent. As a result, cracks may occur in the zeolite membrane and the separation performance (e.g., separation ratio) of the zeolite membrane may deteriorate.

Furthermore, if the supply-side surface area is excessively larger or excessively smaller than the permeation-side surface area, the process of forming the zeolite membrane on the support by hydrothermal synthesis may end with non-uniform supply of a starting material solution to seed crystals that adhere on the support, and this may result in non-uniform growth of the zeolite membrane and accordingly a decrease in flux and deterioration in separation performance. Besides, an increasing amount of extension of the zeolite membrane into the surface of the support may reduce the aforementioned flux, or a reducing amount of extension of the zeolite membrane into the surface of the support may result in an insufficient reduction in the difference in thermal expansion between the zeolite membrane and the support. This may cause cracks in the zeolite membrane and deterioration in separation performance. However, conventionally diversified consideration has not been made on the aforementioned ratio between the supply-side surface area and the permeation-side surface area.

### SUMMARY OF THE INVENTION

The present invention is intended for a separation membrane complex, and it is an object of the present invention to provide a separation membrane complex that exhibits a high flux and high separation performance and in which a separation membrane is joined on a support with a reduced difference in thermal expansion.

A separation membrane complex according to one preferable embodiment of the present invention includes a porous support, and a separation membrane formed on the support and used to separate fluid. A supply/permeation area ratio obtained by dividing a supply-side surface area by a permeation-side surface area is higher than or equal to 1.1 and lower than or equal to 5.0, the supply-side surface area being an area of a region of a surface of the separation membrane to which fluid is supplied, the permeation-side surface area being an area of a region of a surface of the support from which fluid that has permeated through the separation membrane and the support flows off.

According to the present invention, it is possible to provide a separation membrane complex that exhibits a high flux and high separation performance and in which the separation membrane is joined on the support with a reduced difference in thermal expansion.

Preferably, the separation membrane may have a thickness greater than or equal to 0.05 gm and less than or equal to 50 μm.

Preferably, the separation membrane may be a zeolite membrane.

Preferably, the zeolite membrane may be composed of a maximum 8- or less-membered ring zeolite.

Preferably, the support may include a porous base material, and a porous surface layer provided on the base material and having a smaller mean pore diameter than the base material.

Preferably, the base material may have a mean pore diameter greater than or equal to 1 μm and less than or equal to 50 μm, and the surface layer may have a mean pore diameter greater than or equal to 0.005 μm and less than or equal to 2 μm.

Preferably, the support may further include a porous intermediate layer that is provided between the base material and the surface layer and that has a smaller mean pore diameter than the base material, the base material and the surface layer may be composed primarily of Al₂O₃, and the intermediate layer may include aggregate particles composed primarily of Al₂O₃, and an inorganic binding material that is composed primarily of TiO₂ and that binds the aggregate particles together.

Preferably, the support may have a honeycomb shape in which a plurality of cells, each being a through hole extending in a longitudinal direction, are provided in a column-like body extending in the longitudinal direction.

Preferably, each of the plurality of cells may have a sectional area of 2 mm² or more and 300 mm² or less perpendicular to the longitudinal direction.

Preferably, the plurality of cells may be arranged in a grid in lengthwise and crosswise directions at an end face of the support, the plurality of cells may include a plurality of cell lines arranged in the lengthwise direction, each of the cell lines being a group of cells aligned in a row in the crosswise direction, and the plurality of cell lines may include a mesh-sealed cell line that is a single cell line having mesh-sealed ends on both sides in the longitudinal direction, and an open cell-line group that include two or more and six or less cell lines located adjacent to one side of the mesh-sealed cell line in the lengthwise direction and each having open ends on both sides in the longitudinal direction.

Preferably, the support may include a slit that extends from an outside surface of the support through the mesh-sealed cell line in the crosswise direction.

The present invention is also intended for a separation method. A separation method according to one preferable embodiment of the present invention includes a) preparing the separation membrane complex described above, and b) supplying a mixture of substances that contains a plurality of types of gas or liquid to the separation membrane complex and causing a substance with high permeability in the mixture of substances to permeate through the separation membrane complex and to be separated from the other substances.

Preferably, the mixture of substances may include one or more kinds of substances selected from among hydrogen, helium, nitrogen, oxygen, water, water vapor, carbon monoxide, carbon dioxide, nitrogen oxides, ammonia, sulfur oxides, hydrogen sulfide, sulfur fluoride, mercury, arsine, hydrogen cyanide, carbonyl sulfide, C1 to C8 hydrocarbons, organic acid, alcohol, mercaptans, ester, ether, ketone, and aldehyde.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a perspective view of a separation membrane complex according to one embodiment;

FIG. 2 is an illustration of an end face of the separation membrane complex;

FIG. 3 is a sectional view of the separation membrane complex;

FIG. 4 is an illustration of an end face of the separation membrane complex;

FIG. 5 is a flowchart of production of the separation membrane complex;

FIG. 6 is an illustration of a separation device; and

FIG. 7 is a flowchart of separation of a mixture of substances.

### DETAILED DESCRIPTION

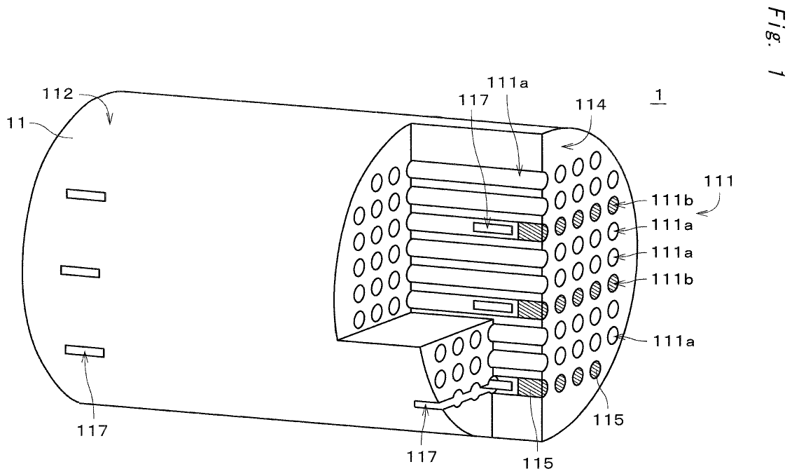

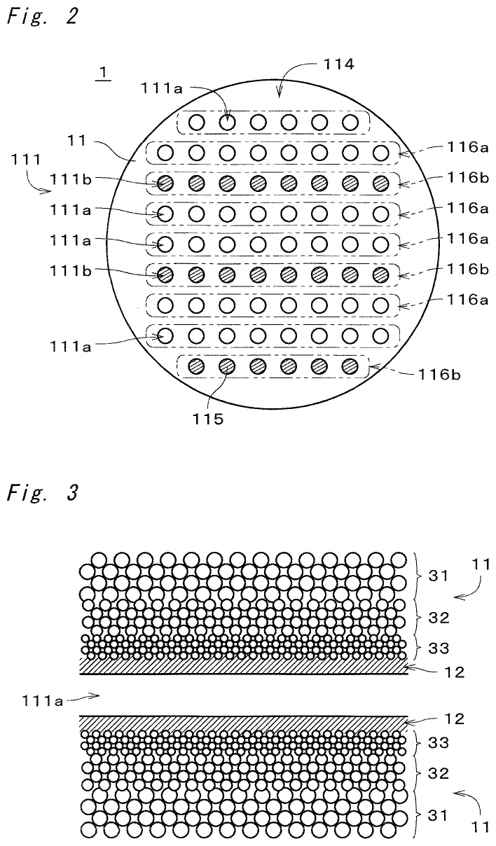

FIG. 1 is a perspective view of a separation membrane complex 1 according to one embodiment of the present invention. In FIG. 1, part of the internal structure of the separation membrane complex 1 is also shown. FIG. 2 is an illustration of an end face 114 of the separation membrane complex 1. FIG. 3 is an enlarged view of part of a longitudinal section of the separation membrane complex 1 and shows an area in the vicinity of a cell 111, which will be described later. The separation membrane complex 1 is used to separate a specific substance from a mixture of substances including a plurality of types of substances.

The separation membrane complex 1 includes a porous support 11 and a zeolite membrane 12 (see FIG. 3) that is a separation membrane formed on the support 11. In FIG. 3, the zeolite membrane 12 is cross-hatched. The zeolite membrane 12 refers to at least a zeolite formed into a membrane on the surface of the support 11, and does not refer to zeolite particles that are merely dispersed in an organic membrane. The zeolite membrane 12 may contain two or more types of zeolites having different structures or different compositions. Note that the separation membrane complex 1 may include a separation membrane other than the zeolite membrane 12.

The support 11 is a porous member that is permeable to gas and liquid. In the example illustrated in FIG. 1, the support 11 is a honeycomb support in which an integrally molded column-like body has a plurality of through holes 111 (hereinafter, also referred to as “cells 111”), each extending in the longitudinal direction of the body (i.e., in approximately the right-left direction in FIG. 1). The support 11 has a plurality of cells 111 formed (sectioned) by a porous partition wall. In the example illustrated in FIG. 1, the support 11 has an approximately column-like outside shape. Each cell 111 may have, for example, an approximately circular cross-sectional shape perpendicular to the longitudinal direction. In the illustration of FIG. 1, the cells 111 have a diameter greater than the actual diameter of the cells 111, and the number of cells 111 is smaller than the actual number of cells 111.

The cells 111 include first cells 111a that have the zeolite membrane 12 formed on the inside surfaces, and second cells 111b that do not have the zeolite membrane 12 formed on the inside surfaces. At both end faces 114 of the support 11 in the longitudinal direction, openings of the second cells 111b are mesh-sealed with mesh sealing members 115. In FIGS. 1 and 2, the mesh sealing members 115 are cross-hatched. On the other hand, openings of the first cells 111a are not mesh-sealed and are open at the both end faces 114 of the support 11 in the longitudinal direction.

In the example illustrated in FIGS. 1 and 2, the cells 111 are arranged in a grid in the lengthwise direction (i.e., the up-down direction in FIG. 2) and the crosswise direction at the end faces 114 of the support 11. In the following description, a group of cells 111 aligned in a row in the crosswise direction (i.e., the right-left direction in FIG. 2) is also referred to as a “cell line.” The cells 111 include a plurality of cell lines arranged in the lengthwise direction. In the example illustrated in FIG. 2, each cell line is composed of either a plurality of first cells 111a or a plurality of second cells 111b.

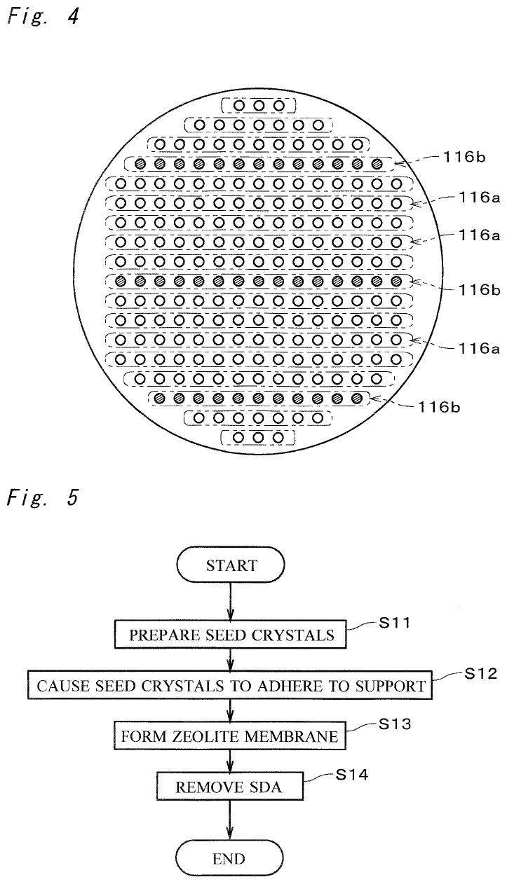

In the example illustrated in FIG. 2, the cell lines are arranged such that a single cell line of second cells 111b (hereinafter, also referred to as a “second cell line 116b”) and two cell lines of first cells 111a (hereinafter, also referred to as “first cell lines 116a”) are alternately arranged adjacent to each other in the lengthwise direction. In FIG. 2, the first cell lines 116a and the second cell lines 116b are each surrounded by a chain double-dashed line (the same applies to FIG. 4 described later). The second cell lines 116b are mesh-sealed cell lines whose both ends in the longitudinal direction are mesh-sealed. The second cells 111b of each second cell line 116b communicate with one another via a slit 117 and communicate with the space outside the support 11 via the slit 117 that extends to an outside surface 112 of the support 11. In other words, the slit 117 extends from the outside surface of the support 11 through the second cell line 116b in the crosswise direction.

The first cell lines 116a are open cell lines whose both ends in the longitudinal direction are open, and two lines of first cells 111a that are located adjacent to one side of one second cell line 116b in the lengthwise direction are referred to as an open cell line group. In other words, the open cell line group refers to a plurality of first cell lines 116a sandwiched between two second cell lines 116b that are located closest to each other in the lengthwise direction. The number of first cell lines 116a that configure one open cell line group is not limited to two, and may be modified in various ways. Preferably, the number of first cell lines 116a that configure one open cell line group may be two or more and six or less. FIG. 4 shows an example in which five first cell lines 116a configure one open cell line group.

The support 11 may have a length of, for example, 100 mm to 2000 mm in the longitudinal direction. The support 11 may have an outside diameter of, for example, 5 mm to 300 mm. A cell-to-cell distance between adjacent cells 111 (i.e., the thickness of the support 11 between the closest portions of adjacent cells 111) may be in the range of, for example, 0.3 mm to 10 mm. The surface roughness (Ra) of the support 11 may, for example, be in the range of 0.1 μm to 5.0 μm and preferably in the range of 0.2 μm to 2.0 μm. The area of a section of each cell 111 perpendicular to the longitudinal direction may, for example, be larger than or equal to 2 mm² and smaller than or equal to 300 mm². In the case where each cell 111 has an approximately circular sectional shape as described above, the diameter of the above section may preferably be in the range of 1.6 mm to 20 mm.

Note that the shapes and sizes of the support 11 and the cells 111 may be modified in various ways. For example, the cells 111 may have an approximately elliptical sectional shape or an approximately polygonal sectional shape. As another alternative, the support 11 may have, for example, a plate-like shape, a tube-like shape, a cylinder-like shape, a column-like shape, or a polygonal column-like shape. In the case where the support 11 has a tube- or cylinder-like shape, the thickness of the support 11 may be in the range of, for example, 0.1 mm to 10 mm.

The material for the support 11 may be any of a variety of substances (e.g., ceramic or metal) as long as the substance has chemical stability during the process of forming the zeolite membrane 12 on the surface. In the present embodiment, the support 11 is formed of a ceramic sintered body. Examples of the ceramic sintered body that is selected as the material for the support 11 include alumina, silica, mullite, zirconia, titania, yttrium, silicon nitride, and silicon carbide. In the present embodiment, the support 11 contains at least one type of substances including alumina, silica, and mullite.

The support 11 may include an inorganic binding material for binding aggregate particles of the ceramic sintered body described above together. The inorganic binding material may be at least one of titania, mullite, easily sinterable alumina, silica, glass frit, clay minerals, or easily sinterable cordierite.

For example, the support 11 may have a multilayer structure in which a plurality of layers having different mean pore diameters are laminated one above another in the thickness direction in the vicinity of the inside surface of each first cell 111a, which is an open cell. In the example illustrated in FIG. 3, the support 11 includes a porous base material 31, a porous intermediate layer 32 formed on the base material 31, and a porous surface layer 33 formed on the intermediate layer 32. That is, the surface layer 33 is provided indirectly on the base material 31 via the intermediate layer 32. The intermediate layer 32 is provided between the base material 31 and the surface layer 33. The surface layer 33 configures the inside surface of each first cell 111a of the support 11, and the zeolite membrane 12 is formed on the surface layer 33. The surface layer 33 may have a thickness of, for example, 1 μm to 100 μm. The intermediate layer 32 may have a thickness of, for example, 100 μm to 500 μm. Note that the intermediate layer 32 and the surface layer 33 may be or may not be provided on the inside surface of each second cell 111b. Also, the intermediate layer 32 and the surface layer 33 may be or may not be provided on the outside surface 112 and the end faces 114 of the support 11.

The surface layer 33 has a mean pore diameter smaller than the mean pore diameters of the intermediate layer 32 and the base material 31. The mean pore diameter of the intermediate layer 32 is smaller than the mean pore diameter of the base material 31. The mean pore diameter of the base material 31 may, for example, be greater than or equal to 1 μm and less than or equal to 70 μm. The intermediate layer 32 may, for example, have a mean pore diameter greater than or equal to 0.1 μm and less than or equal to 10 μm. The surface layer 33 may, for example, have a mean pore diameter greater than or equal to 0.005 μm and less than or equal to 2 μm. The mean pore diameters of the base material 31, the intermediate layer 32, and the surface layer 33 may be measured by, for example, a mercury porosimeter, a perm porosimeter, or a nano-perm porosimeter.

The porosity of the surface layer 33 is lower than the porosity of the intermediate layer 32 and the porosity of the base material 31. The porosity of the intermediate layer 32 is lower than the porosity of the base material 31. The porosity of the base material 31 may, for example, be higher than or equal to 25% and lower than or equal to 50%. The porosity of the intermediate layer 32 may, for example, be higher than or equal to 15% and lower than or equal to 70%. The porosity of the surface layer 33 may, for example, be higher than or equal to 15% and lower than or equal to 70%. For example, the porosities of the base material 31, the intermediate layer 32, and the surface layer 33 may be measured by a mercury porosimeter, a perm porosimeter, or a nano-perm porosimeter.

The base material 31, the intermediate layer 32, and the surface layer 33 may be formed of the same material, or may be formed of different materials. For example, the base material 31 and the surface layer 33 may be composed primarily of Al₂O₃. The intermediate layer 32 includes aggregate particles composed primarily of Al₂O₃ and an inorganic binding material composed primarily of TiO₂. In the present embodiment, the aggregate particles of the base material 31, the intermediate layer 32, and the surface layer 33 are substantially composed of only Al₂O₃. The base material 31 may include an inorganic binding material such as glass.

The aggregate particles of the surface layer 33 have an average particle diameter smaller than the average particle diameter of the aggregate particles in the intermediate layer 32. The aggregate particles of the intermediate layer 32 have an average particle diameter smaller than the average particle diameter of the base material 31. The average particle diameters of the aggregate particles of the base material 31, the intermediate layer 32, and the surface layer 33 may be measured by, for example, laser diffractometry.

The mesh sealing members 115 may be formed of a material similar to the materials for the base material 31, the intermediate layer 32, and the surface layer 33. The porosity of the mesh sealing members 115 may be in the range of, for example, 25% to 50%.

The zeolite membrane 12 is formed on the inside surface of each first cell 111a, which is an open cell (i.e., on the surface layer 33), and covers approximately the entire inside surface. The zeolite membrane 12 is a porous membrane having microscopic pores. The zeolite membrane 12 can be used as a separation membrane that separates a specific substance from a mixture of substances including a plurality of types of substances, using the molecular-sieving function. The zeolite membrane 12 is less permeable to the other substances than to the specific substance. In other words, the permeance of the zeolite membrane 12 to the other substances is lower than the permeance thereof to the aforementioned specific substance. Note that the zeolite membrane 12 is not provided on the inside surface of each second cell 111b.

The zeolite membrane 12 may, for example, have a thickness greater than or equal to 0.05 μm and less than or equal to 50 μm, preferably greater than or equal to 0.1 μm and less than or equal to 20 μm, and more preferably greater than or equal to 0.5 μm and less than or equal to 10 μm. Increasing the thickness of the zeolite membrane 12 improves separation performance. Reducing the thickness of the zeolite membrane 12 increases permeance. The surface roughness (Ra) of the zeolite membrane 12 may, for example, be less than or equal to 5 μm, preferably less than or equal to 2 μm, more preferably less than or equal to 1 μm, and yet more preferably less than or equal to 0.5 μm. The zeolite membrane 12 may have a pore diameter of, for example, 0.2 nm to 1 nm. The pore diameter of the zeolite membrane 12 is smaller than the mean pore diameter of the surface layer 33 of the support 11.

In the case where the zeolite membrane 12 is composed of a maximum n-membered ring zeolite, the minor axis of an n-numbered ring pore is assumed to be the pore diameter of the zeolite membrane 12. In the case where the zeolite has a plurality of types of n-membered ring pores where n is the same number, the minor axis of an n-membered ring pore that has a largest minor axis is assumed to be the pore diameter of the zeolite membrane 12. Note that the n-membered ring refers to a portion in which n oxygen atoms constitute the framework of a pore and each oxygen atom is bonded to a T atom described later to form a cyclic structure. The n-membered ring also refers to a portion that forms a through hole (channel), and does not refer to a portion that fails to form a through hole. The n-membered ring pore refers to a small pore formed of an n-membered ring. From the viewpoint of improving selectivity, the aforementioned zeolite membrane 12 may preferably contain a maximum 8- or less-membered ring zeolite (e.g., 6- or 8-membered ring zeolite).

The pore diameter of the zeolite membrane is uniquely determined by the framework structure of the zeolite and can be obtained from a value disclosed in “Database of Zeolite Structures” by the International Zeolite Association, [online], from the Internet <URL:http://www.iza-structure.org/databases/>.

There are no particular limitations on the type of the zeolite of the zeolite membrane 12, and examples of the zeolite include AEI-, AEN-, AFN-, AFV-, AFX-, BEA-, CHA-, DDR-, ERI-, ETL-, FAU- (X-type, Y-type), GIS-, IHW-, LEV-, LTA-, LTJ-, MEL-, MFI-, MOR-, PAU-, RHO-, SOD-, and SAT-type zeolites. In the case where the zeolite is an 8-membered ring zeolite, examples of the zeolite include AEI-, AFN-, AFV-, AFX-, CHA-, DDR-, ERI-, ETL-, GIS-, IHW-, LEV-, LTA-, LTJ-, RHO-, and SAT-type zeolites. In the present embodiment, the zeolite of the zeolite membrane 12 is an DDR-type zeolite.

The zeolite of the zeolite membrane 12 contains aluminum (Al), phosphorus (P), and a tetravalent element as T atoms (i.e., atoms located in the center of an oxygen tetrahedron (TO₄) constituting the zeolite). The tetravalent element may preferably be one or more types of elements selected from among silicon (Si), germanium (Ge), titanium (Ti), and zirconium (Zr), more preferably one or more types of elements selected from among Si and Ti, and yet more preferably Si. In the case where the tetravalent element is Si, the zeolite of the zeolite membrane 12 may, for example, be an SAPO-type zeolite that contains Si, Al, and P as the T atoms, an MAPSO-type zeolite that contains magnesium (Mg), Si, Al, and. P as the T atoms, or a ZnAPSO-type zeolite that contains zinc (Zn), Al, and P as the T atoms. Some of the T atoms may be replaced by other elements. The zeolite of the zeolite membrane 12 may contain alkali metal. Examples of the alkali metal include sodium (Na) and potassium (K).

The zeolite membrane 12 may contain, for example, Si. For example, the zeolite membrane 12 may contain any two or more of Si, Al, and P. The zeolite membrane 12 may contain alkali metal. The alkali metal may, for example, be sodium (Na) or potassium (K). In the case where the zeolite membrane 12 contains Si atoms and Al atoms, the Si/Al ratio in the zeolite membrane 12 may, for example, be higher than or equal to one and lower than or equal to a hundred thousand. The Si/Al ratio is the molar ratio of Si elements to Al elements contained in the zeolite membrane 12. The Si/Al ratio may preferably be higher than or equal to 5, more preferably higher than or equal to 20, and yet more preferably higher than or equal to 100. A higher Si/Al ratio is more preferable because the zeolite membrane 12 can achieve higher heat resistance to heat and acids. The Si/Al ratio in the zeolite membrane 12 may be adjusted by adjusting, for example, the compounding ratio of an Si source and an Al source in a starting material solution described later.

In the case where the difference in partial pressure of CO₂ between the supply side and permeation side of the zeolite membrane 12 is 1.5 MPa, the CO₂ permeance (permeance) of the zeolite membrane 12 at a temperature of 20° C. to 400° C. may, for example, be higher than or equal to 100 nmol/(m²·Pa·sec), and the ratio (permeance ratio) between CO₂ permeance and CH₄ leakage in the zeolite membrane 12 at a temperature of 20° C. to 400° C. may, for example, be higher than or equal to 25. In the case where the aforementioned difference in partial pressure of CO₂ is 0.2 MPa, the aforementioned permeance may, for example, be higher than or equal to 200 nmol/(m²·Pa·sec), and the aforementioned permeance ratio may, for example, be higher than or equal to 60.

Next, one example of the procedure for producing the separation membrane complex 1 will be described with reference to FIG. 5. In the production of the separation membrane composite 1, first, seed crystals that are used to form the zeolite membrane 12 are synthesized and prepared (step S11). In the synthesis of the seed crystals, starting materials such as an Si source and other materials such as a structure-directing agent (hereinafter, also referred to as an “SDA”) are dissolved or dispersed in a solvent to prepare a starting material solution of the seed crystals. Then, the starting material solution is subjected to hydrothermal synthesis, and resultant crystals are washed and dried to obtain zeolite powder. The zeolite powder may be used as-is as the seed crystals, or the zeolite powder may be processed into the seed crystals by, for example, pulverization.

Then, a dispersion liquid obtained by dispersing the seed crystals in a solvent (e.g., water or alcohol such as ethanol) is poured into the first cells 111a of the support 11. For example, the support 11 may be placed on a base such that the longitudinal direction of the support 11 becomes approximately parallel to the direction of gravity, and the dispersion liquid may be poured from the upper opening of each first cell 111a so that the seed crystals in the dispersion liquid adhere to inside surface of the first cell 111a (step S12). The dispersion liquid poured into the first cells 111a is discharged from the lower openings of the first cells 111a. Preferably, step S12 may be repeated multiple times (e.g., 2 times to 10 times). More preferably, the support 11 may be turned upside down during the multiple repetition of step S12. This produces a seed-crystal-deposited support with the seed crystals adhering uniformly to the inside surface of each first cell 111a. Note that any other technique may be used to cause the seed crystals to adhere to the inside surfaces of the first cells 111a.

Then, the support 11 with the seed crystals adhering thereto is immersed in a starting material solution. The starting material solution may be prepared by, for example, dissolving substances such as an Si source and an SDA in a solvent. The solvent in the starting material solution may, for example, be water or alcohol such as ethanol. The SDA contained in the starting material solution may, for example, be an organic substance. The SDA may, for example, be 1-adamantanamine.

Then, the zeolite is grown by hydrothermal synthesis using the aforementioned seed crystals as nuclei, so as to form the zeolite membrane 12 on the inside surface of each first cell 111a of the support 11 (step S13). The temperature at the time of the hydrothermal synthesis may preferably be in the range of 120 to 200° C. and may, for example, be 160° C. The hydrothermal synthesis time may preferably be in the range of 10 to 100 hours and may, for example, be 30 hours.

When the hydrothermal synthesis is completed, the support 11 and the zeolite membrane 12 are washed with deionized water. After the washing, the support 11 and the zeolite membrane 12 may be dried at, for example, 80° C. After the drying of the support 11 and the zeolite membrane 12, the zeolite membrane 12 is subjected to heat treatment (i.e., firing) so as to almost completely burn and remove the SDA in the zeolite membrane 12 and to cause microscopic pores in the zeolite membrane 12 to come through the zeolite membrane 12. In this way, the aforementioned separation membrane complex 1 is obtained (step S14).

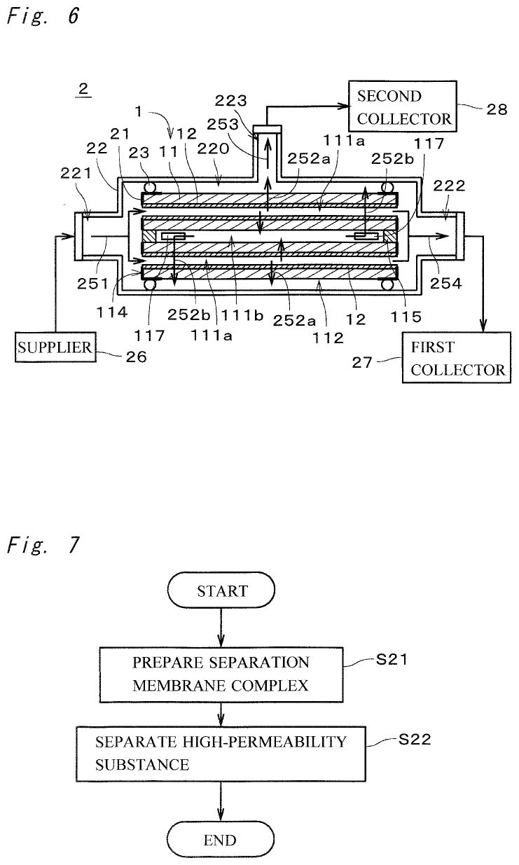

Next, the separation of a mixture of substances by using the separation membrane complex 1 will be described with reference to FIGS. 6 and 7. FIG. 6 is a sectional view of a separation device 2. In FIG. 6, a section of the separation membrane complex 1 is illustrated in simplified and conceptual manner in order to facilitate understanding of the drawing. FIG. 7 is a flowchart of the separation of a mixture of substances using the separation device 2.

The separation device 2 supplies a mixture of substances including a plurality of types of fluid (i.e., gas or liquid) to the separation membrane complex 1 and causes a substance with high permeability in the mixture of substances to permeate through the separation membrane complex 1 and to be separated from the mixture of substances. For example, the separation device 2 may perform this separation for the purpose of extracting a substance with high permeability (hereinafter, also referred to as a “high-permeability substance”) from the mixture of substances or for the purpose of concentrating a substance with lower permeability (hereinafter, also referred to as a “low-permeability substance”).

The mixture of substances (i.e., a fluid mixture) may be a mixed gas that includes a plurality of types of gas, may be a mixed solution that includes a plurality of types of liquid, or may be a gas-liquid two-phase fluid that includes both gas and liquid.

For example, the mixture of substances may include one or more types of substances selected from among hydrogen (H₂), helium (He), nitrogen (N₂), oxygen (O₂), water (H₂O), water vapor (H₂O), carbon monoxide (CO), carbon dioxide (CO₂), nitrogen oxides, ammonia (NH₃), sulfur oxides, hydrogen sulfide (H₂S), sulfur fluorides, mercury (Hg), arsine (AsH₃), hydrogen cyanide (HCN), carbonyl sulfide (COS), C1 to C8 hydrocarbons, organic acid, alcohol, mercaptans, ester, ether, ketone, and aldehyde. The aforementioned high-permeability substance may, for example, be one or more types of substances selected from among Co₂, NH₃, and H₂O and may preferably be H₂O.

Nitrogen oxides are compounds of nitrogen and oxygen. For example, the aforementioned nitrogen oxides may be gas called NOₓ such as nitrogen monoxide (NO), nitrogen dioxide (NO₂), nitrous oxide (also referred to as dinitrogen monoxide) (N₂O), dinitrogen trioxide (N₂O₃), dinitrogen tetroxide (N₂O₄), or dinitrogen pentoxide (N₂O₅).

Sulfur oxides are compounds of sulfur and oxygen. For example, the aforementioned sulfur oxides may be gas called SOₓ such as sulfur dioxide (SO₂) or sulfur trioxide (SO₃).

Sulfur fluorides are compounds of fluorine and sulfur. For example, the aforementioned sulfur fluorides may be disulfur difluoride (F—S—S—F, S═SF₂), sulfur difluoride (SF₂), sulfur tetrafluoride (SF₄), sulfur hexafluoride (SF₆), or disulfur decafluoride (S₂F₁₀).

C1 to C8 hydrocarbons are hydrocarbons that contain one or more and eight or less carbon atoms. C3 to C8 hydrocarbons each may be any of a linear-chain compound, a side-chain compound, and a cyclic compound. C2 to C8 hydrocarbons each may be either of a saturated hydrocarbon (i.e., where double bonds and triple bonds are not located in molecules) and an unsaturated hydrocarbon (i.e., where double bonds and/or triple bonds are located in molecules). C1 to C4 hydrocarbons may, for example, be methane (CH₄), ethane (C₂H₆), ethylene (C₂H₄), propane (C₃H₈), propylene (C₃H₆), normal butane (CH₃(CH₂)₂CH₃), isobutene (CH(CH₃)₃), 1-butene (CH₂═CHCH₂CH₃), 2-butene (CH₃CH═CHCH₃), or isobutene (CH₂═C(CH₃)₂).

The aforementioned organic acid may, for example, be carboxylic acid or sulfonic acid. The carboxylic acid may, for example, be formic acid (CH₂O₂), acetic acid (C₂H₄O₂), oxalic acid (C₂H₂O₄), acrylic acid (C₃H₄O₂), or benzoic acid (C₆H₅COOH). The sulfonic acid may, for example, be ethane sulfonic acid (C₂H₆O₃S). The organic acid may be a chain compound, or may be a cyclic compound.

The aforementioned alcohol may, for example, be methanol (CH₃OH), ethanol (C₂H₅OH), isopropanol (2-propanol) (CH₃CH(OH)CH₃), ethylene glycol (CH₂(OH)CH₂(OH)), or butanol (C₄H₉OH).

Mercaptans are organic compounds with terminal sulfur hydrides (SH) and are substances called also thiol or thioalcohol. The aforementioned mercaptans may, for example, be methyl mercaptan (CH₃SH), ethyl mercaptan (C₂H₅SH), or 1-propane thiol (C₃H₇SH).

The aforementioned ester may, for example, be formic acid ester or acetic acid ester.

The aforementioned ether may, for example, be dimethyl ether ((CH₃)₂O), methyl ethyl ether (C₂H₅OCH₃), or diethyl ether ((C₂H₅)₂O).

The aforementioned ketone may, for example, be acetone ((CH₃)₂CO), methyl ethyl ketone (C₂H₅COCH₃), or diethyl ketone ((C₂H₅)₂CO).

The aforementioned aldehyde may, for example, be acetaldehyde (CH₃CHO), propionaldehyde (C₂H₅CHO), or butanal (butyraldehyde) (C₃H₇CHO).

The following description is given on the assumption that the mixture of substances that is subjected to the separation by the separation device 2 is a mixed gas that includes a plurality of types of gas.

The separation device 2 includes the separation membrane complex 1, a sealer 21, an outer cylinder 22, two seal members 23, a supplier 26, a first collector 27, and a second collector 28. The separation membrane complex 1, the sealer 21, and the seal members 23 are placed in the outer cylinder 22. The supplier 26, the first collector 27, and the second collector 28 are disposed outside the outer cylinder 22 and connected to the outer cylinder 22. In FIG. 6, the zeolite membrane 12 of the separation membrane complex 1 is cross-hatched.

The sealer 21 is a member that is attached to both ends of the support 11 in the longitudinal direction (i.e., in the left-right direction in FIG. 1) to cover and seal the both end faces 114 of the support 11 in the longitudinal direction and part of the outside surface 112 of the support 11 in the vicinity of the both end faces 114. The sealer 21 prevents the inflow and outflow of liquid from the both end faces 114 of the support 11. The sealer 21 may, for example, be a plate-like member formed of glass or resin. In the present embodiment, the sealer 21 is a glass seal having a thickness of 30 μm to 50 μm. The material and shape of the sealer 21 may be appropriately changed. Note that the sealer 21 has a plurality of openings that overlap with the plurality of first cells 111a of the support 11, so that the both ends of each first cell 111a of the support 11 in the longitudinal direction are not covered with the sealer 21. This allows the inflow and outflow of fluid from the both ends into and out of the first cells 111a.

The outer cylinder 22 is an approximately cylindrical tube-like member. For example, the outer cylinder 22 may be formed of stainless steel or carbon steel. The longitudinal direction of the outer cylinder 22 is approximately parallel to the longitudinal direction of the separation membrane complex 1. One end of the outer cylinder 22 in the longitudinal direction (i.e., the end on the left side in FIG. 6) has a supply port 221, and the other end thereof has a first exhaust port 222. The side face of the outer cylinder 22 has a second exhaust port 223. The supply port 221 is connected to the supplier 26. The first exhaust port 222 is connected to the first collector 27. The second exhaust port 223 is connected to the second collector 28. The internal space of the outer cylinder 22 is an enclosed space that is isolated from the space around the outer cylinder 22.

The two seal members 23 are arranged around the entire circumference between the outside surface 112 of the separation membrane complex 1 and the inside surface of the outer cylinder 22 in the vicinity of the both ends of the separation membrane complex 1 in the longitudinal direction. Each seal member 23 is an approximately ring-shaped member formed of a material that is impermeable to gas and liquid. For example, the seal members 23 may be O-rings formed of resin having flexibility. The seal members 23 are in tight contact with the outside surface 112 of the separation membrane complex 1 and the inside surface of the outer cylinder 22 along the entire circumference. In the example illustrated in FIG. 6, the seal members 23 are in tight contact with the outside surface of the sealer 21 and indirectly in tight contact with the outside surface of the separation membrane complex 1 via the sealer 21. The space between the seal members 23 and the outside surface of the separation membrane complex 1 and the space between the seal members 23 and the inside surface of the outer cylinder 22 are sealed so as to almost or completely disable the passage of gas and liquid.

The supplier 26 supplies the mixed gas to the internal space of the outer cylinder 22 via the supply port 221. The supplier 26 may include, for example, a pressure mechanism such as a blower or a pump that pumps the mixed gas toward the outer cylinder 22. The pressure mechanism may include, for example, a temperature regulator and a pressure regulator that control respectively the temperature and pressure of the mixed gas supplied to the outer cylinder 22. The first collector 27 and the second collector 28 may include, for example, a reservoir that stores gas delivered from the outer cylinder 22, or a blower or a pump that transfers this gas.

In the separation of the mixed gas, first, the separation membrane complex 1 is prepared (step S21 in FIG. 7). Specifically, the separation membrane complex 1 is mounted on the inside of the outer cylinder 22. Then, the mixed gas including a plurality of types of gas having different permeability to the zeolite membrane 12 is supplied from the supplier 26 to the inside of the outer cylinder 22 (specifically, the space on the left side of the left end face 114 of the separation membrane complex 1) as indicated by an arrow 251. For example, the mixed gas may be composed primarily of CO₂ and CH₄. The mixed gas may also include gas other than CO₂ and CH₄. The pressure of the mixed gas supplied from the supplier 26 to the inside of the outer cylinder 22 (i.e., initial pressure) may be in the range of, for example, 0.1 MPa to 20.0 MPa. The temperature of the mixed gas supplied from the supplier 26 may be in the range of, for example, 10° C. to 250° C.

The mixed gas supplied from the supplier 26 to the outer cylinder 22 flows into each first cell 111a of the separation membrane complex 1. In the mixed gas, gas with high permeability, i.e., a high-permeability substance, permeates through the zeolite membrane 12 and the support 11 from the first cells 111a and is exhausted from the outside surface 112 of the separation membrane complex 1 to a separated space 220 around the separation membrane complex 1 as indicated by an arrow 252a. The separated space 220 is an approximately cylindrical space located radially outward of the outside surface 112 of the separation membrane complex 1. The high-permeability substance flowing from the first cells 111a through the zeolite membrane 12 and the support 11 into the second cells 111b is guided to the outside surface 112 of the separation membrane complex 1 through the slits 117 and led out to the separated space 220 as indicated by an arrow 252b. Note that the high-permeability substance flowing from the first cells 111a into the second cells 111b may be led out to the separated space 220 through the support 11 without passing through the slits 117.

In this way, the high-permeability substance is led out to the separated space 220 through the zeolite membrane 12. Accordingly, the high-permeability substance (e.g., CO₂) is separated from the other substances such as gas with low permeability in the mixed gas, i.e., a low-permeability substance (e.g., CH₄) (step S22). As described above, since the end faces 114 of the support 11 are covered with the sealer 21, the separation membrane complex 1 can prevent or suppress the entry of the mixed gas that contains low-permeability substances into the inside of the support 11 via the end faces 114 and accordingly the entry of the mixed gas into the separated space 220 without causing the mixed gas to permeate through the zeolite membrane 12. The gas led out from the outside surface 112 of the separation membrane complex 1 (hereinafter, referred to as a “permeated substance”) is guided via the second exhaust port 223 to the second collector 28 and collected by the second collector 28 as indicated by an arrow 253 in FIG. 6. The permeated substance may further include, in addition to the aforementioned high-permeability substance, a low-permeability substance that has permeated through the zeolite membrane 12.

In the mixed gas, gas (hereinafter, also referred to as a “non-permeated substance”) other than the substance that has permeated through the zeolite membrane 12 and the support 11 is guided via the first exhaust port 222 to the first collector 27 and collected by the first collector 27 as indicated by an arrow 254. The non-permeated substance may further include, in addition to the aforementioned low-permeability substance, a high-permeability substance that has not permeated through the zeolite membrane 12. The non-permeated substance collected by the first collector 27 may, for example, be circulated into the supplier 26 and supplied again to the inside of the outer cylinder 22.

In the following description, the area of a region of the surface of the zeolite membrane 12 in the separation membrane complex 1 to which fluid such as the aforementioned mixed gas is supplied is referred to as a “supply-side surface area Ss.” In the example described above, the supply-side surface area Ss corresponds to a total surface area of the zeolite membrane 12 formed on the inside surfaces of the first cells 111a. In other words, the supply-side surface area Ss is a total area of exposed surfaces of the zeolite membrane 12 that are exposed to the inside of the first cells 111a. In the case where some regions of the zeolite membrane 12 (e.g., regions located in the vicinity of the end portions of the separation membrane complex 1 in the longitudinal direction) are covered with another structure such as the sealer 21 and the aforementioned fluid is not supplied to these regions, the surface areas of these regions are not included in the supply-side surface area Ss.

In the following description, the area of a region of the surface of the support 11 from which fluid such as a high-permeability substance that has permeated through the zeolite membrane 12 and the support 11 flows off is referred to as a “permeation-side surface area St.” In the example described above, the permeation-side surface area St corresponds to a total surface area of the outside surface 112 of the support 11, the inside surfaces of the slits 117, and the inside surfaces of the second cells 111b. Since the both end faces 114 of the support 11 in the longitudinal direction are covered with the sealer 21 and therefore fluid that has permeated through the zeolite membrane 12 and the support 11 does not flow off, the surface areas of the end faces 114 are not included in the permeation-side surface area St.

In the case where some of the regions including the outside surface 112 of the support 11, the inside surfaces of the slits 117, and the inside surfaces of the second cells 111b are covered with another structure such as the sealer 21 and accordingly fluid does not flow off from these regions, the surface areas of these regions are not included in the permeation-side surface area St. For example, the regions of the outside surface 112 of the support 11 that are located in the vicinity of the both end portions in the longitudinal direction are covered with the sealer 21 and therefore the areas of these regions are not included in the permeation-side surface area St. Moreover, the regions of the inside surfaces of the second cells 111b that are located in the vicinity of the both end portions in the longitudinal direction are covered with the mesh sealing members 115, and therefore the areas of these regions are also not included in the permeation-side surface area St. In the case where the support 11 has no slits 117 and does not provide communication between the second cells 111b and the outside surface 112 of the support 11, the permeation-side surface area St is equivalent to the surface area of the outside surface 112 of the support 11 (except the regions covered with the sealer 21).

In the following description, the value obtained by dividing the supply-side surface area Ss by the permeation-side surface area St is referred to as a “supply/permeation area ratio.” The supply/permeation area ratio is higher than or equal to 1.1 and lower than or equal to 5.0. The supply/permeation area ratio may preferably be higher than or equal to 2.0 and more preferably higher than or equal to 3.0. The supply/permeation area ratio may also preferably be lower than or equal to 4.5 and more preferably lower than or equal to 4.0.

In the case where the supply/permeation area ratio is excessively low, the amount of the mixed gas supplied per unit time to the zeolite membrane 12 of each first cell 111a may decrease and this may result in reduced efficiency of processing for separating the mixed gas by the separation membrane complex 1 and increased processing cost. As described above, by setting the supply/permeation area ratio to be higher than or equal to 1.1, the separation membrane complex 1 is capable of increasing the amount of the mixed gas supplied per unit time to the zeolite membrane 12 and thereby increasing the flux of the high-permeability substance that permeates through the zeolite membrane 12. As a result, it is possible to improve the efficiency of the processing for separating the mixed gas and to suppress an increase in processing cost required for the separation processing.

In the case where the supply/permeation area ratio is excessively high or excessively low, a large difference may occur between SDA emission from the surface side of the zeolite membrane 12 (i.e., vaporized SDA emission) and SDA emission from the rear surface side of the zeolite membrane 12 during the process of burning and removing the SDA from the zeolite membrane 12 in the production of the separation membrane complex 1 (step S14 in FIG. 5). The surface side of the zeolite membrane 12 as used herein refers to the exposed surface side of the zeolite membrane 12 that is exposed to the inside of the first cells 111a, and the rear surface side of the zeolite membrane 12 refers to the joint surface side of the zeolite membrane 12 that is joined to the support 11. As described above, if the difference in SDA emission between the surface side and rear surface side of the zeolite membrane 12 increases, the time required for removal of the SDA may become non-uniform in the thickness direction of the zeolite membrane 12, and this may result in an insufficient or uneven removal of the SDA such as SDA residues in part of the zeolite membrane 12 (in the area where SDA emission is small). If the SDA is to be removed completely, there is a need to increase the heating temperature of the zeolite membrane 12, but in this case, problems such as cracks in the zeolite membrane 12 may occur due to a difference in thermal expansion. As a result, the separation performance (e.g., separation ratio) of the separation membrane complex 1 may deteriorate. Besides, a decrease in yield may occur during the production of the separation membrane complex 1.

As described above, the separation membrane complex 1 is capable of reducing the difference in SDA emission between the surface and rear surface sides of the zeolite membrane 12 by setting the supply/permeation area ratio to be higher than or equal to 1.1 and lower than or equal to 5.0. As a result, it is possible to prevent or suppress an insufficient or uneven removal of the SDA from the zeolite membrane 12. It is also possible to prevent or suppress the occurrence of damage such as cracks due to excessive heating of the zeolite membrane 12. This improves the separation performance of the separation membrane complex 1. This also improves a yield during the production of the separation membrane complex 1.

In the separation membrane complex 1, if the supply/permeation area ratio is excessively high or excessively low, the amount of starting materials supplied from the starting material solution that has permeated through the partition wall of the first cells 111a to the seed crystals adhering to the surface of the support 11 (hereinafter, this amount is also referred to as the “starting-material supply”) may deviate from an appropriate range during the process of forming the zeolite membrane 12 on the support 11 by hydrothermal synthesis in the production of the separation membrane complex 1 (step S13 in FIG. 5). For example, an excessively low supply/permeation area ratio may cause an excessive increase in starting-material supply from the starting material solution that has permeated through the partition walls and accordingly cause an excessive increase in the amount of extension of the zeolite membrane 12 into the support 11. As a result, the flux of the high-permeability substance in the separation membrane complex 1 may decrease. Moreover, an excessively high supply/permeation area ratio may cause an excessive decrease in starting-material supply from the starting material solution that has permeated through the partition wall and accordingly cause an excessive decrease in the amount of extension of the zeolite membrane 12 into the support 11. As a result, the reduction in the difference in thermal expansion between the zeolite membrane 12 and the support 11 may not be enough, and cracks may occur in the zeolite membrane 12 during the process of burning and removing the SDA (step S14) and may deteriorate the separation performance.

As described above, the separation membrane complex 1 is capable of setting the supply/permeation area ratio to be higher than or equal to 1.1 and lower than or equal to 5.0, so that the starring-material supply to the seed crystals on the rear surface side of the zeolite membrane 12 falls within an appropriate range. As a result, the amount of extension of the zeolite membrane 12 into the support 11 can fall within a favorable range. Accordingly, it is possible to increase the flux in the zeolite membrane 12 and to join the zeolite membrane 12 on the support 11 with a reduced difference in thermal expansion.

The supply/permeation area ratio described above may be adjusted by various methods. For example, the supply/permeation area ratio may be changed to a relatively large value by changing the number of first cell lines 116a that configure one open cell line group. Moreover, the supply/permeation area ratio may be lowered by providing two or more second cell lines 116b, which are mesh-sealed cell lines, between two open cell line groups. The supply/permeation area ratio may also be changed by changing the cell-to-cell distance and thereby changing the number of first cells 111a included in each first cell line 116a. Similarly, the supply/permeation area ratio may also be changed by changing the cell-to-cell distance and thereby changing the number of second cells 111b included in each second cell line 116b. The supply/permeation area ratio may also be changed by changing the sectional areas of the first cells 111a and/or the second cells 111b. The supply/permeation area ratio may also be changed by changing the length of the slits 117.

Next, the relationship between the characteristics of the separation membrane complex 1 and the supply/permeation area ratio in the separation membrane complex 1 according to Examples 1 to 16 with reference to Tables 1 to 8. The same is also applied to Comparative Examples 1 to 11.

TABLE 1

| | Support | | | | | |
|---|---|---|---|---|---|---|
| | | Number of Lines | Mean Pore | | | |
| | | in Open Cell | Diameter of | Zeolite Membrane | | Supply/ |
| | | Line Group | Surface Layer | | Thickness | Permeation |
| | Structure | (Lines) | (μm) | Type | (μm) | Area Ratio |
| Example 1 | Honeycomb Shape | 2 | 0.05 | DDR-Type | 0.5 | 1.50 |
| Example 2 | Honeycomb Shape | 5 | 0.05 | DDR-Type | 0.5 | 4.29 |
| Example 3 | Honeycomb Shape | 2 | 0.005 | DDR-Type | 0.5 | 1.50 |
| Example 4 | Honeycomb Shape | 2 | 0.05 | DDR-Type | 1 | 1.50 |
| Example 5 | Honeycomb Shape | 2 | 0.05 | DDR-Type | 1 | 2.00 |
| Example 6 | Honeycomb Shape | 3 | 0.05 | DDR-Type | 1 | 2.75 |
| Example 7 | Honeycomb Shape | 4 | 0.05 | DDR-Type | 1 | 3.29 |
| Example 8 | Honeycomb Shape | 5 | 0.05 | DDR-Type | 1 | 4.00 |
| Example 9 | Honeycomb Shape | 5 | 0.05 | DDR-Type | 1 | 4.29 |
| Example 10 | Honeycomb Shape | 5 | 0.05 | AEI-Type | 1 | 4.29 |
| Example 11 | Honeycomb Shape | 5 | 0.05 | MFI-Type | 1 | 4.29 |
| Example 12 | Honeycomb Shape | 6 | 0.05 | DDR-Type | 1 | 4.80 |
| Example 13 | Honeycomb Shape | 2 | 1.2 | DDR-Type | 38 | 1.50 |
| Example 14 | Honeycomb Shape | 5 | 1.2 | DDR-Type | 38 | 4.29 |
| Example 15 | Cylindrical Tube-like | — | 0.05 | DDR-Type | 1 | 1.30 |
| | Shape | | | | | |
| Example 16 | Cylindrical Tube-like | — | 0.05 | DDR-Type | 1 | 4.00 |
| | Shape | | | | | |
| Comparative | Honeycomb Shape | 1 | 0.05 | DDR-Type | 0.5 | 1.07 |
| Example 1 | | | | | | |
| Comparative | Honeycomb Shape | 6 | 0.05 | DDR-Type | 0.5 | 5.40 |
| Example 2 | | | | | | |
| Comparative | Honeycomb Shape | 1 | 0.05 | DDR-Type | 1 | 1.07 |
| Example 3 | | | | | | |
| Comparative | Honeycomb Shape | 6 | 0.05 | DDR-Type | 1 | 5.40 |
| Example 4 | | | | | | |
| Comparative | Honeycomb Shape | 6 | 0.01 | DDR-Type | 1 | 5.40 |
| Example 5 | | | | | | |
| Comparative | Honeycomb Shape | 6 | 0.05 | AEI-Type | 1 | 5.40 |
| Example 6 | | | | | | |
| Comparative | Honeycomb Shape | 6 | 0.05 | MFI-Type | 1 | 5.40 |
| Example 7 | | | | | | |
| Comparative | Honeycomb Shape | 1 | 1.2 | DDR-Type | 38 | 1.07 |
| Example 8 | | | | | | |
| Comparative | Honeycomb Shape | 6 | 1.2 | DDR-Type | 38 | 5.40 |
| Example 9 | | | | | | |
| Comparative | Cylindrical Tube-like | — | 0.05 | DDR-Type | 1 | 1.08 |
| Example 10 | Shape | | | | | |
| Comparative | Cylindrical Tube-like | — | 0.05 | DDR-Type | 1 | 6.00 |
| Example 11 | Shape | | | | | |

TABLE 2

| | Characteristics | |
|---|---|---|
| | Ratio of | Ratio of |
| | CO₂ Flux | Separation Factor |
| Example 1 | 1.00 | 1.00 |
| Example 2 | 1.14 | 1.19 |
| Comparative Example 1 | 0.33 | 0.28 |
| Comparative Example 2 | * 1.55 | 0.05 |

* With Cracks

TABLE 3

| | Characteristics | |
|---|---|---|
| | Ratio of | Ratio of |
| | CO₂ Flux | Separation Factor |
| Example 4 | 1.00 | 1.00 |
| Example 5 | 1.07 | 1.11 |
| Example 6 | 1.12 | 1.19 |
| Example 7 | 1.28 | 1.37 |
| Example 8 | 1.38 | 1.35 |
| Example 9 | 1.35 | 1.37 |
| Example 12 | 1.22 | 1.19 |
| Comparative Example 3 | 0.24 | 0.45 |
| Comparative Example 4 | * 1.66 | 0.05 |
| Comparative Example 5 | * 1.87 | 0.04 |

* With Cracks

TABLE 4

| | Characteristics | |
|---|---|---|
| | Ratio of | Ratio of |
| | CO₂ Flux | Separation Factor |
| Example 13 | 1.00 | 1.00 |
| Example 14 | 1.27 | 1.15 |
| Comparative Example 8 | 0.82 | 0.65 |
| Comparative Example 9 | * 25.64 | 0.01 |

* With Cracks

TABLE 5

| | Characteristics | |
|---|---|---|
| | Ratio of | Ratio of |
| | CO₂ Flux | Separation Factor |
| Example 1 | 1.00 | 1.00 |
| Example 3 | 0.82 | 0.72 |

| | Characteristics | |
|---|---|---|
| | Ratio of | Ratio of |
| | CO₂ Flux | Separation Factor |
| Example 10 | 1.00 | 1.00 |
| Comparative Example 6 | * 2.13 | 0.05 |

* With Cracks

TABLE 7

| | Characteristics | |
|---|---|---|
| | Ratio of | Ratio of |
| | CO₂ Flux | Separation Factor |
| Example 11 | 1.00 | 1.00 |
| Comparative Example 7 | ※ 1.10 | 0.75 |

* With Cracks

TABLE 8

| | Characteristics | |
|---|---|---|
| | Ratio of | Ratio of |
| | CO₂ Flux | Separation Factor |
| Example 15 | 1.00 | 1.00 |
| Example 16 | 0.89 | 0.95 |
| Comparative Example 10 | 0.56 | 0.28 |
| Comparative Example 11 | ※ 1.19 | 0.02 |

* With Cracks

In Examples 1 to 16, the CO₂ flux and the separation factor for the separation membrane complex 1 were measured as the characteristics of the separation membrane complex 1 by changing the shape of the support 11, the pore diameter of the surface layer 33, the type and thickness of the zeolite membrane 12, and the supply/permeation area ratio in various ways. The same was done for Comparative Examples 1 to 11.

The CO₂ fluxes in Tables 2 to 8 were measured by a method described below and then compared relatively with the CO₂ fluxes in the other examples and the comparative examples, using the CO₂ flux in the uppermost line of each table as a reference of 1.00 (i.e., divided by the CO₂ flux in the uppermost line of each example). In the measurement of the CO₂ flux, CO₂ was first supplied to the separation membrane complex 1, using the separation device 2 described above, and the flow rate of CO₂ that has permeated through the zeolite membrane 12 and the support 11 was measured by a mass flow meter. Then, this flow rate was divided by the surface area of the zeolite membrane 12 so as to obtain the aforementioned CO₂ flux (L/(min·m²)).

The separation factors in Tables 2 to 8 are indicators that indicate the separation performance of the zeolite membrane 12, and a higher separation factor indicates higher separation performance. These separation factors were measured by a method described below and then compared relatively with the separation factors in the other examples and the comparative examples, using the separation factor in the uppermost line of each table as a reference of 1.00 (i.e., divided by the separation factor in the uppermost line of each example). In the measurement of the separation factor, the separation device 2 described above was first used to supply a 25° C. mixed gas that contained 50% by volume of CO₂ and 50% by volume of CH₄ to the separation membrane complex 1 at a total pressure of 0.4 MPa (i.e., a partial pressure of 0.2 MPa for each of CO₂ and CH₄). Then, the flow rate of the gas that has permeated through the zeolite membrane 12 and the support 11 was measured by a mass flow meter. The gas that has permeated through the zeolite membrane 12 and the support 11 was also subjected to component analysis using a gas chromatograph. Then, the separation factor was obtained from the CO₂/CH₄ permeance ratio (i.e., the ratio of the flux per unit pressure difference, per unit area, and per unit time).

The separation factors in Tables 2 to 8 are also indicators that indicate whether cracks have occurred in the zeolite membrane 12. Specifically, if the reduction in the difference in thermal expansion between the zeolite membrane 12 and the support 11 is not enough, relatively large cracks occur in the zeolite membrane 12 due to this difference during the process of burning and removing the SDA in step S14 described above, and accordingly the separation factor decreases.

In Example 1, the support 11 had a honeycomb structure, and the number of lines included in one open cell line group (i.e., the number of first cell lines 116a that configure one open cell line group) was two, which was the same as the number in the example illustrated in FIG. 2. The surface layer 33 of the support 11 had a mean pore diameter of 0.05 μm. The type of the zeolite of the zeolite membrane 12 was the DDR type (8-membered ring), and the zeolite membrane 12 had a thickness of 0.5 μm. The supply/permeation area ratio of the separation membrane complex 1 was set to 1.50.

Example 2 was the same as Example 1, except that the number of lines included in one open cell line group was five (see FIG. 4) and the supply/permeation area ratio was set to 4.29.

Example 3 was the same as Example 1, except that the surface layer 33 had a mean pore diameter of 0.005 μm.

Example 4 was the same as Example 1, except that the zeolite membrane 12 had a thickness of 1 μm.

Example 5 was the same as Example 4, except that the supply/permeation area ratio was set to 2.00.

Example 6 was the same as Example 4, except that the number of lines included in one open cell line group was three and the supply/permeation area ratio was set to 2.75.

Example 7 was the same as Example 4, except that the number of lines included in one open cell line group was four and the supply/permeation area ratio was set to 3.29.

Example 8 was the same as Example 4, except that the number of lines included in one open cell line group was five (see FIG. 4) and the supply/permeation area ratio was set to 4.00.

Example 9 was the same as Example 8, except that the supply/permeation area ratio was set to 4.29.

Example 10 was the same as Example 9, except that the type of the zeolite of the zeolite membrane 12 was the AEI type (8-membered ring).

Example 11 was the same as Example 9, except that the type of the zeolite of the zeolite membrane 12 was the MFI type (10-membered ring).

Example 12 was the same as Example 4, except that the number of lines included in one open cell line group was six and the supply/permeation area ratio was set to 4.80.

Example 13 was the same as Example 1, except that the surface layer 33 had a mean pore diameter of 1.2 μm and the zeolite membrane 12 had a thickness of 38 μm.

Example 14 was the same as Example 13, except that the number of lines included in one open cell line group was five (see FIG. 4) and the supply/permeation area ratio was set to 4.29.

In Example 15, the support 11 had a circular tube-like structure, and the surface layer 33 of the support 11 had a mean pore diameter of 0.05 μm. The type of the zeolite of the zeolite membrane 12 was the DDR type (8-membered ring), and the zeolite membrane 12 had a thickness of 1 μm. The supply/permeation area ratio of the separation membrane complex 1 was set to 1.30.

Example 16 was the same as Example 15, except that the supply/permeation area ratio was set to 4.00.

Comparative Example 1 was the same as Example 1, except that the number of lines included in one open cell line group was one, and the supply/permeation area ratio was set to 1.07.

Comparative Example 2 was the same as Example 1, except that the number of lines included in one open cell line group was six and the supply/permeation area ratio was set to 5.40.

Comparative Example 3 was the same as Example 4, except that the number of lines included in one open cell line group was one and the supply/permeation area ratio was set to 1.07.

Comparative Example 4 was the same as Example 4, except that the number of lines included in one open cell line group was six, and the supply/permeation area ratio was set to 5.40.

Comparative Example 5 was the same as Comparative Example 4, except that the surface layer 33 had a mean pore diameter of 0.01 μm.

Comparative Example 6 was the same as Comparative Example 4, except that the type of the zeolite of the zeolite membrane 12 was the AEI type (8-membered ring).

Comparative Example 7 was the same as Comparative Example 4, except that the type of the zeolite of the zeolite membrane 12 was the MFI type (10-membered ring).

Comparative Example 8 was the same as Comparative Example 3, except that the surface layer 33 had a mean pore diameter of 1.2 μm and the zeolite membrane 12 had a thickness of 38 μm.

Comparative Example 9 was the same as Comparative Example 8, except that the number of lines included in one open cell line group was six and the supply/permeation area ratio was set 5.40.

Comparative Example 10 was the same as Example 15, except that the supply/permeation area ratio was set to 1.08.

Comparative Example 11 was the same as Example 15, except that the supply/permeation area ratio was set to 6.00.

As shown in Table 2, the comparison between Examples 1 and 2 and Comparative Examples 1 and 2, all using the zeolite membrane 12 with a thickness of 0.5 μm, indicates that the supply/permeation area ratios in Examples 1 and 2 were in the range of 1.50 to 4.29 (i.e., higher than or equal to 1.1 and lower than or equal to 5.0), whereas the supply/permeation area ratio in Comparative Example 1 was 1.07 (i.e., lower than 1.1) and the supply/permeation area ratio in Comparative Example 2 was 5.40 (i.e., higher than 5.0). Note that Examples 1 and 2 and Comparative Examples 1 and 2 were the same in the structure of support 11 (honeycomb shape), the mean pore diameter of the support 11, and the type and thickness (0.5 μm) of the zeolite membrane 12.

In Examples 1 and 2 in which the supply/permeation area ratios were higher than or equal to 1.1 and lower than or equal to 5.0, the ratios of the CO₂ flux (i.e., the ratios using Example 1 as a reference) were in the range of 1.00 to 1.14 and the ratios of the separation factor (i.e., the ratios using Example 1 as a reference) were in the range of 1.00 to 1.19. In Example 1, the CO₂ flux and the separation factor were 458 L/(min·m²) and 151, respectively. On the other hand, in Comparative Example 1 in which the supply/permeation area ratio was lower than 1.1, the ratio of the CO₂ flux was 0.33 and lower than those in Examples 1 and 2. In Comparative Example 1, the separation performance was lower than in Examples 1 and 2 because the ratio of the separation factor was 0.28 and low. In Comparative Example 2 in which the supply/permeation area ratio was higher than 5.0, the separation performance was lower than in Examples 1 and 2 because the ratio of the separation factor was 0.05 and low. In Comparative Example 2 in which the supply/permeation area ratio was higher than 5.0, the separation performance was lower than in Examples 1 and 2 because the ratio of the separation factor was 0.05 and low. Moreover, in Comparative Example 2, the ratio of the CO₂ flux was high in proportion to the membrane thickness because cracks had occurred in the zeolite membrane 12 during production. This indicates that, in Comparative Example, 2, the reduction in the difference in thermal expansion between the zeolite membrane 12 and the support 11 was not enough as compared to that in Examples 1 and 2.

As shown in Table 3, the comparison between Examples 4 to 9 and 12 and Comparative Examples 3 to 5, all using the zeolite membrane 12 with a thickness of 1 μm, indicates that the supply/permeation area ratios in Examples 4 to 9 and 12 were in the range of 1.50 to 4.80 (i.e., higher than or equal to 1.1 and lower than or equal to 5.0), whereas the supply/permeation area ratio in Comparative Example 3 was 1.07 (i.e., lower than 1.1), and the supply/permeation area ratios in Comparative Examples 4 and 5 were 5.40 (i.e., higher than 5.0). Note that Examples 4 to 9 and 12 and Comparative Examples 3 and 4 were the same in the structure of the support 11 (honeycomb shape), the mean pore diameter of the surface layer 33, the type and thickness (1 μm) of the zeolite membrane 12. Comparative Example 5 was also the same in the structure (honeycomb shape) of the support 11 and the type and thickness of the zeolite membrane 12.

In Examples 4 to 9 and 12 in which the supply/permeation area ratios were higher than or equal to 1.1 and lower than or equal to 5.0, the ratios of the CO₂ flux (i.e., the ratios using Example 4 as a reference) were in the range of 1.00 to 1.38, and the ratios of the separation factor (i.e., the ratios using Example 4 as a reference) were in the range of 1.00 to 1.37. In Example 4, the CO₂ flux and the separation factor were 258 L/(min·m²) and 194, respectively. On the other hand, in Comparative Example 3 in which the supply/permeation area ratio was lower than 1.1, the ratio of the CO₂ flux was 0.24 and lower than those in Examples 4 to 9 and 12. In Comparative Example 3, the separation performance was lower than in Examples 4 to 9 and 12 because the ratio of the separation factor was 0.45 and low. In Comparative Examples 4 and 5 in which the supply/permeation area ratios were higher than 5.0, the separation performance was lower than in Examples 4 to 9 and 12 because the ratios of the separation factor were in the range of 0.04 to 0.05 and low. Moreover, in Comparative Examples 4 and 5, the ratios of the CO₂ flux were high in proportion to the membrane thickness because cracks had occurred in the zeolite membrane 12 during production. This indicates that, in Comparative Examples 4 and 5, the reduction in the difference in thermal expansion between the zeolite membrane 12 and the support 11 was not enough as compared to that in Examples 4 to 9 and 12.

As shown in Table 4, the comparison between Examples 13 and 14 and Comparative Examples 8 and 9, all using the zeolite membrane 12 with a thickness of 38 μm, indicates that the supply/permeation area ratios in Examples 13 and 14 were in the range of 1.50 to 4.29 (i.e., higher than or equal to 1.1 and lower than or equal to 5.0), whereas the supply/permeation area ratio in Comparative Example 8 was 1.07 (i.e., lower than 1.1) and the supply/permeation area ratio in Comparative Example 9 was 5.40 (i.e., higher than 5.0). Note that Examples 13 and 14 and Comparative Examples 8 and 9 were the same in the structure (honeycomb shape) of the support 11, the mean pore diameter of the surface layer 33, and the type and thickness (38 μm) of the zeolite membrane 12.

In Examples 13 and 14 in which the supply/permeation area ratios were higher than or equal to 1.1 and lower than or equal to 5.0, the ratios of the CO₂ flux (i.e., the ratios using Example 13 as a reference) were in the range of 1.00 to 1.27, and the ratios of the separation factor (i.e., the ratios using Example 13 as a reference) were in the range of 1.00 to 1.15. In Example 13, the CO₂ flux and the separation factor were 11 L/(min·m²) and 127, respectively. On the other hand, in Comparative Example 8 in which the supply/permeation area ratio was lower than 1.1, the ratio of the CO₂ flux was 0.82 and lower than in Examples 13 and 14. Moreover, in Comparative Example 8, the separation performance was lower than in Examples 13 and 14 because the ratio of the separation factor was 0.65 and low. In Comparative Example 9 in which the supply/permeation area ratio was higher than 5.0, the separation performance was lower than in Examples 13 and 14 because the ratio of the separation factor was 0.01 and low. Moreover, in Comparative Example 9, the ratio of the CO₂ flux was high in proportion to the membrane thickness because cracks had occurred in the zeolite membrane 12 during production. This indicates that, in Comparative Example 9, the reduction in the difference in thermal expansion between the zeolite membrane 12 and the support 11 was not enough as compared to that in Examples 13 and 14.

As shown in Table 5, focusing now on Examples 1 and 3, the surface layers 33 in Examples 1 and 3 had mean pore diameters of 0.05 μm to 0.005 μm, which were included in a range higher than or equal to 0.005 μm and lower than or equal to 2 μm. The mean pore diameter of the surface layer 33 in Example 3 was the lower limit of the range. Note that Examples 1 and 3 were the same in the structure of the support 11, the type and thickness of the zeolite membrane 12, and the supply/permeation area ratio. In Example 3, the ratio of the CO₂ flux using Example 1 as a reference was 0.82, and the ratio of the separation factor was 0.72. In Example 3, since the mean pore diameter of the surface layer 33 was smaller than in Example 1, it can be thought that the amount of extension of the zeolite membrane 12 into the surface of the support 11 decreased and this resulted in an insufficient reduction in the difference in thermal expansion between the zeolite membrane 12 and the support 11. Accordingly, it can be thought that, in Example 3, the separation factor was lowered than that in Example 1 because slight cracks had occurred in part of the zeolite membrane 12 during the process of burning and removing the SDA in step S14.

As shown in Table 6, the comparison between Example 10 and Comparative Example 6, both using the AEI-type zeolite as the zeolite of the zeolite membrane 12, indicates that the supply/permeation area ratio in Example 10 was 4.29 (i.e., higher than or equal to 1.1 and lower than or equal to 5.0), whereas the supply/permeation area ratio in Comparative Example 6 was 5.40 (i.e., higher than 5.0). Note that Example 10 and Comparative Example 6 were the same in the structure of the support 11 (honeycomb shape), the mean pore diameter of the surface layer 33, and the type (AEI type) and thickness of the zeolite membrane 12.

In Comparative Example 6 in which the supply/permeation area ratio was higher than 5.0, the separation performance was lower than in Example 10 because the ratio of the separation factor (i.e., the ratio Example 10 as a reference) was 0.05 and low. Moreover, in Comparative Example 6, the ratio of the CO₂ flux was high in proportion to the membrane thickness because cracks had occurred in the zeolite membrane 12 during production. This indicates that, in Comparative Example 6, the reduction in the difference in thermal expansion between the zeolite membrane 12 and the support 11 was not enough as compared to that in Example 10. In Example 10, the CO₂ flux and the separation factor were 181 L/(min·m²) and 41, respectively.

As shown in Table 7, the comparison between Example 11 and Comparative Example 7, both using the MFI-type zeolite as the zeolite of the zeolite membrane 12, indicates that the supply/permeation area ratio in Example 11 was 4.29 (i.e., higher than or equal to 1.1 and lower than or equal to 5.0), whereas the supply/permeation area ratio in Comparative Example 7 was 5.40 (i.e., higher than 5.0). Note that Examples 11 and Comparative Example 7 were the same in the structure (honeycomb shape) of the support 11, the mean pore diameter of the surface layer 33, and the type (MFI type) and thickness of the zeolite membrane 12.

In Comparative Example 7 in which the supply/permeation area ratio was higher than 5.0, the separation performance was lower than in Example 11 because the ratio of the separation factor (i.e., the ratio using Example 11 as a reference) was 0.75 and low. Moreover, in Comparative Example 7, the ratio of the CO₂ flux was high in proportion to the membrane thickness because cracks had occurred in the zeolite membrane 12 during production. This indicates that, in Comparative Example 7, the reduction in the difference in thermal expansion between the zeolite membrane 12 and the support 11 was not enough as compared to that in Example 11. In Example 11, the CO₂ flux and the separation factor were 759 L/(min·m²) and 4, respectively.

As shown in Table 8, the comparison between Examples 15 and 16 and Comparative Examples 10 and 11, all using the support 11 with a cylindrical tube-like structure, indicates that the supply/permeation area ratios in Examples 15 and 16 were in the range of 1.30 to 4.00 (i.e., higher than or equal to 1.1 and lower than or equal to 5.0), whereas the supply/permeation area ratio in Comparative Example 10 was 1.08 (i.e., lower than 1.1) and the supply/permeation area ratio in Comparative Example 11 was 6.00 (i.e., higher than 5.0). Note that Examples 15 and 16 and Comparative Examples 10 and 11 were the same in the structure (cylindrical tube-like shape) of the support 11, the mean pore diameter of the surface layer 33, and the type and thickness of the zeolite membrane 12.

In Examples 15 and 16 in which the supply/permeation area ratios were higher than or equal to 1.1 and lower than or equal to 5.0, the ratios of the CO₂ flux (i.e., the ratios using Example 15 as a reference) were in the range of 0.89 to 1.00, and the ratios of the separation factor (i.e., the ratios using Example 15 as a reference) were in the range of 0.95 to 1.00. In Example 15, the CO₂ flux and the separation factor were 351 L/(min·m²) and 258, respectively. On the other hand, in Comparative Example 10 in which the supply/permeation area ratio was lower than 1.1, the ratio of the CO₂ flux was 0.56 and lower than those in Examples 15 and 16. In Comparative Example 10, the separation performance was lower than in Examples 15 and 16 because the ratio of the separation factor was 0.28 and low. In Comparative Example 11 in which the supply/permeation area ratio was higher than 5.0, the separation performance was lower than in Examples 15 and 16 because the ratio of the separation factor was 0.02 and low. Moreover, in Comparative Example 11, the ratio of the CO₂ flux was high in proportion to the membrane thickness because cracks had occurred in the zeolite membrane 12 during production. This indicates that, in Comparative Example 11, the reduction in the difference in thermal expansion between the zeolite membrane 12 and the support 11 was not enough as compared to that in Examples 15 and 16.

As described above, the separation membrane complex 1 includes the porous support 11 and the separation membrane (in the above-described example, the zeolite membrane 12) formed on the support 11 and used to separate fluid. The supply/permeation area ratio obtained by dividing the supply-side surface area Ss by the permeation-side surface area St is higher than or equal to 1.1 and lower than or equal to 5.0, the supply-side surface area being the area of the region of the surface of the separation membrane to which fluid is supplied, the permeation-side surface area being the area of the region of the surface of the support 11 from which fluid that has permeated through the separation membrane and the support 11 flows off. Accordingly, as shown in Tables 2 to 4, it is possible to provide the separation membrane complex 1 that exhibits a high flux and high separation performance and in which the separation membrane is joined on the support 11 with a reduced difference in thermal expansion. It is also possible to improve a yield in the production of the separation membrane complex 1.

As described above, the separation membrane may preferably have a thickness greater than or equal to 0.05 μm and less than or equal to 50 μm. In this case, as shown in Tables 2 to 4, it is possible to favorably achieve both an increase in the flux of the separation membrane complex 1 and an improvement in separation performance.

As described above, the support 11 may preferably include the porous base material 31 and the porous surface layer 33 provided on the base material 31 and having a mean pore diameter smaller than that of the base material 31. In this case, it is possible to increase the strength of the support 11 and to favorably form a thin separation membrane on the support 11.

Preferably, the base material 31 may have a mean pore diameter greater than or equal to 1 μm and less than or equal to 50 μm, and the surface layer 33 may have a mean pore diameter greater than or equal to 0.005 μm and less than or equal to 2 μm. In this case, it is possible to further increase the strength of the support 11 and to more favorably form a thin separation membrane. Besides, when the separation membrane is formed by hydrothermal synthesis (step S13), it is possible to cause the starting-material supply to the seed crystals on the rear surface side of the separation membrane that is to be formed, to fall within a favorable range. As a result, it is possible to cause the amount of extension of the separation membrane into the support 11 to fall within a more favorable range. Accordingly, as shown in Table 5, it is possible to increase the flux of the separation membrane and to allow the separation membrane to be joined on the support 11 with a reduced difference in thermal expansion.

Preferably, the support 11 may further include the porous intermediate layer 32 provided between the base material 31 and the surface layer 33 and having a smaller mean pore diameter than the base material 31. The base material 31 and the surface layer 33 contain Al₂O₃ as their primary component, and the intermediate layer 32 contains aggregate particles composed primarily of Al₂O₃ and an inorganic binding material composed primarily of TiO₂ and for binding the aggregate particles together. Accordingly, it is possible to prevent or suppress damage to the separation membrane and the support 11 due to, for example, heat in the process of burning and removing the SDA from the separation membrane (step S14).

The separation membrane described above may preferably be the zeolite membrane 12. When the separation membrane is composed of zeolite crystals having relatively small pore diameters as described above, it is possible to favorably achieve selective permeation of substances that have small molecular sizes and that permeate through the membrane and to efficiently separate such substances from a mixture of substances.

More preferably, the zeolite membrane 12 may be composed of a maximum 8- or less-membered ring zeolite. In this case, it is possible to favorably achieve selective permeation of substances such as H₂ or CO₂ that have small molecular sizes and that permeate through the membrane and to efficiently separate such substances from a mixture of substances (see Examples 9 to 11).

As described above, the support 11 may preferably have a honeycomb shape in which a column-like body extending in the longitudinal direction has a plurality of cells 111, each being a through hole extending in the longitudinal direction. In this case, the area of the separation membrane per unit volume of the separation membrane complex 1 can be increased. As a result, it is possible to further increase the flux of the separation membrane complex 1. It is also possible to achieve the separation membrane complex 1 with high strength while increasing the area of the separation membrane.

As described above, the area of the section of each cell 111 that is perpendicular to the longitudinal direction may preferably be larger than or equal to 2 mm² and smaller than or equal to 300 mm². Setting this area to 2 mm² or more allows the dispersion liquid containing the seed crystals to easily flow into the cells 111. Moreover, setting this area to 300 mm² or less shortens the time required for the solvent in the dispersion liquid flowing into the cells 111 to be discharged through the support 11 to the outside of the cells 111. As a result, it is possible to favorably apply the seed crystals to the inside surfaces of the cells 111 (in the above-described example, the inside surfaces of the first cells 111a).

Preferably, the cells 111 may be arranged in a grid in the lengthwise and crosswise directions at the end faces 114 of the support 11. The cells 111 include a plurality of cell lines arranged in the lengthwise direction, each cell line being composed of a group of cells arranged in a line in the crosswise direction. The cell lines include a mesh-sealed cell line (i.e., a second cell line 116b) that is a single cell line whose both ends in the longitudinal direction are mesh-shielded, and a group of open cell lines (i.e., two or more and six or less first cell lines 116a) whose both ends in the longitudinal direction are open and that include two or more and six or less cell lines located adjacent to one side of the mesh-sealed cell line in the lengthwise direction. This facilitates the operation of setting the supply/permeation area ratio to be higher than or equal to 1.1 and lower than or equal to 5.0. Accordingly, it is possible to favorably achieve the honeycomb separation membrane complex 1 that exhibits a high flux and high separation performance.

More preferably, the support 11 may include the slits 117 that extend from the outside surface 112 of the support 11 through the aforementioned mesh-sealed cell line (i.e., the second cell line 116b) in the crosswise direction. Accordingly, it is possible to easily lead the fluid that has flowed from the inside of the first cells 111a into the second cell lines 116b through the separation membrane and the support 11 (e.g., a high-permeability substance) to the outside of the separation membrane complex 1.

The separation method described above includes the step of preparing the separation membrane complex 1 (step S21), and the step of supplying a mixture of substances including a plurality of types of gas or liquid to the separation membrane complex 1 and causing a substance with high permeability (i.e., a high-permeability substance) in the mixture of substances to permeate through the separation membrane complex 1 and to be separated from the other substances (step S22). Accordingly, as described above, it is possible to increase the flux in the separation of the mixture of substances and to improve the separation performance.

This separation method is in particular suitable when the mixture of substances includes one or more types of substances selected from among hydrogen, helium, nitrogen, oxygen, water, water vapor, carbon monoxide, carbon dioxide, nitrogen oxides, ammonia, sulfur oxides, hydrogen sulfide, sulfur fluoride, mercury, arsine, hydrogen cyanide, carbonyl sulfide, C1 to C8 hydrocarbons, organic acid, alcohol, mercaptans, ester, ether, ketone, and aldehyde.

The separation membrane complex 1 and the separation method described above may be modified in various ways.

For example, in the support 11, the area of the section of each cell 111 that is perpendicular to the longitudinal direction may be smaller than 2 mm² or may be larger than 300 mm².

The number of first cell lines 116a that configure one open cell line group (i.e., the number of lines of first cells 111a that are sandwiched between two second cell lines 116b located in the closest vicinity in the lengthwise direction) may be one, or may be six or more. Moreover, two or more second cell lines 116b, which are mesh-sealed cell lines, may be provided in succession in the lengthwise direction.

As described above, the slits 117 may be omitted from the support 11. Each cell line of the support 11 may include a mixture of first and second cells 111a and 111b. The cells 111 do not necessarily have to be arranged in a grid in the lengthwise and crosswise directions at the end faces 114 of the support 11, and the arrangement of the cells 111 may be modified in various ways. For example, focusing on two cell lines located adjacent to each other in the longitudinal direction, cells 111 included in one of the two cell lines and cells 111 included in the other cell line may be arranged in a staggered configuration in the crosswise direction, and each cell 111 included in the one cell line may be located at approximately the center in the crosswise direction of two adjacent cells 111 included in the other cell line. In this case, it is possible to reduce the interval in the lengthwise direction between the two cell lines while maintaining the cell interval between the two cell lines.

The features such as the materials and mean pore diameters of the base material 31, the intermediate material 32, and the surface layer 33 of the support 11 or the average particle diameter of the aggregate particles are not limited to the examples described above, and may be modified in various ways. The support 11 may include a plurality of intermediate layers 32 having different mean pore diameters or the like and laminated between the base material 31 and the surface layer 33. Moreover, the surface layer 33 or the intermediate layer 32 may be omitted from the support 11. In the case where the intermediate layer 32 is omitted, the surface layer 33 is provided directly on the base material 31.

Alternatively, the surface layer 33 and the intermediate layer 32 may be omitted from the support 11, and the support 11 may have, for example, a uniform mean pore diameter and a uniform average particle diameter of the aggregate particles. In this case, the support 11 may have a mean pore diameter of, for example, 0.01 μm to 70 μm and preferably 0.05 μm to 25 μm. Referring to the pore size distribution of the support 11, D5 may be in the range of, for example, 0.01 μm to 50 μm, D50 may be in the range of, for example, 0.05 μm to 70 μm, and D95 may be in the range of, for example, 0.1 μm to 2000 μm. The porosity of the support 11 may be in the range of, for example, 20% to 60%.

As described above, the shape of the support 11 is not limited to the honeycomb shape and may be modified in various ways. For example, the zeolite membrane 12 may be formed on the outside surface of the approximately cylindrical support 11. Even in this case, if the supply/permeation area ratio is set to be higher than or equal to 1.1 and lower than or equal to 5.0 as described above, it is possible to provide the separation membrane complex 1 that exhibits a high flux and high separation performance and in which the zeolite membrane 12 is joined on the support 11 with a reduced difference in thermal expansion. Moreover, setting the supply/permeation area ratio to be higher than or equal to 1.1 prevents an excessive decrease in the radial thickness of the support 11 and prevents or suppresses a reduction in the strength of the separation membrane complex 1.

The zeolite membrane 12 may be composed of a maximum 8- or less-membered ring zeolite. As described above, the separation membrane complex 1 may include the zeolite membrane 12 that is formed of any of various types of zeolites.

The separation membrane complex 1 may further include, in addition to the support 11 and the zeolite membrane 12, a functional membrane or a protection membrane that is laminated on the zeolite membrane 12. Such a functional or protection membrane may be an inorganic membrane such as a zeolite membrane, a silica membrane, or a carbon membrane, or may be an organic membrane such as a polyimide membrane or a silicone membrane. Note that the area of a region of the surface of the zeolite membrane 12 that is covered with the functional or protection membrane described above is also included in the supply-side surface area Ss described above.

In the separation membrane complex 1, a separation membrane other than the zeolite membrane 12 (e.g., an inorganic membrane or an organic membrane described above) may be formed on the support 11, instead of the zeolite membrane 12. In this case, as described above, it is also preferable that the separation membrane may have a thickness greater than or equal to 0.1 μm and less than or equal to 50 μm. Note that, irrespective of the type of the separation membrane, the thickness of the separation membrane may be less than 0.1 μm, or may be greater than 50 μm.

The separation device 2 and the separation method described above may be used to separate a substance other than the substance described above by way of example from the mixture of substances.

The configurations of the above-described preferred embodiments and variations may be appropriately combined as long as there are no mutual inconsistencies.

While the invention has been shown and described in detail, the foregoing description is in all aspects illustrative and not restrictive. It is therefore to be understood that numerous modifications and variations can be devised without departing from the scope of the invention.

### INDUSTRIAL APPLICABILITY

The separation membrane complex according to the present invention may, for example, be applicable as a gas separation membrane, and may further be applicable as a membrane for use in various fields, such as a separation membrane for separating substances other than gas or an adsorption membrane for adsorbing various substances.

### REFERENCE SIGNS LIST

1 separation membrane complex

11 support

12 zeolite membrane

31 base material

32 intermediate layer

33 surface layer

111 cell

111a first cell

111b second cell

112 outside surface (of support)

114 end face (of support)

116a first cell line

116b second cell line

117 slit

S11 to S14, S21 to S22 step

1. A separation membrane complex comprising:
a porous support; and
a separation membrane formed on said support and used to separate fluid,
wherein a supply/permeation area ratio obtained by dividing a supply-side surface area by a permeation-side surface area is higher than or equal to 1.1 and lower than or equal to 5.0, the supply-side surface area being an area of a region of a surface of said separation membrane to which fluid is supplied, and the permeation-side surface area being an area of a region of a surface of said support from which fluid that has permeated through said separation membrane and said support flows off,
said separation membrane is a zeolite membrane, and
said zeolite membrane is composed of a maximum 8- or less-membered ring zeolite.

2. The separation membrane complex according to claim 1, wherein
said separation membrane has a thickness greater than or equal to 0.05 μm and less than or equal to 50 μm.

3. The separation membrane complex according to claim 1, wherein
said support includes:
a porous base material; and
a porous surface layer provided on said base material and having a smaller mean pore diameter than said base material.

4. The separation membrane complex according to claim 3, wherein
said base material has a mean pore diameter greater than or equal to 1 μm and less than or equal to 50 μm, and
said surface layer has a mean pore diameter greater than or equal to 0.005 μm and less than or equal to 2 μm.

5. The separation membrane complex according to claim 3, wherein
said support further includes a porous intermediate layer that is provided between said base material and said surface layer and that has a smaller mean pore diameter than said base material,
said base material and said surface layer are composed primarily of Al₂O₃, and
said intermediate layer includes:
aggregate particles composed primarily of Al₂O₃; and
an inorganic binding material that is composed primarily of TiO₂ and that binds said aggregate particles together.

6. The separation membrane complex according to claim 1, wherein
said support has a honeycomb shape in which a plurality of cells, each being a through hole extending in a longitudinal direction, are provided in a column-like body extending in the longitudinal direction.

7. The separation membrane complex according to claim 6, wherein
each of said plurality of cells has a sectional area of 2 mm² or more and 300 mm² or less perpendicular to the longitudinal direction.

8. The separation membrane complex according to claim 6, wherein
said plurality of cells are arranged in a grid in lengthwise and crosswise directions at an end face of said support,
said plurality of cells include a plurality of cell lines arranged in the lengthwise direction, each of the cell lines being a group of cells aligned in a row in the crosswise direction, and
said plurality of cell lines include:
a mesh-sealed cell line that is a single cell line having mesh-sealed ends on both sides in the longitudinal direction; and
an open cell-line group that include two or more and six or less cell lines located adjacent to one side of said mesh-sealed cell line in the lengthwise direction and each having open ends on both sides in the longitudinal direction.

9. The separation membrane complex according to claim 8, wherein
said support includes a slit that extends from an outside surface of said support through said mesh-sealed cell line in the crosswise direction.

10. A separation method comprising:
a) preparing the separation membrane complex according to claim 1; and
b) supplying a mixture of substances that contains a plurality of types of gas or liquid to said separation membrane complex and causing a substance with high permeability in said mixture of substances to permeate through said separation membrane complex and to be separated from the other substances.

11. The separation method according to claim 10, wherein
said mixture of substances includes one or more kinds of substances selected from among hydrogen, helium, nitrogen, oxygen, water, water vapor, carbon monoxide, carbon dioxide, nitrogen oxides, ammonia, sulfur oxides, hydrogen sulfide, sulfur fluoride, mercury, arsine, hydrogen cyanide, carbonyl sulfide, C1 to C8 hydrocarbons, organic acid, alcohol, mercaptans, ester, ether, ketone, and aldehyde.
